(19) United States
(12) Patent Application Publication
(10) Pub. No.: US 2015/0335566 A1
(43) Pub. Date: Nov. 26, 2015

(54) STYLING AGENTS HAVING MAXIMUM HOLD

(71) Applicant: Henkel AG & Co. KGaA, Duesseldorf (DE)

(72) Inventors: Thorsten Knappe, Schenefeld (DE); Helga van Flodrop, Duesseldorf (DE)

(73) Assignee: Henkel AG & Co. KGaA

(21) Appl. No.: 14/816,161

(22) Filed: Aug. 3, 2015

Related U.S. Application Data
(63) Continuation of application No. 14/096,269, filed on Dec. 4, 2013, now abandoned.
Application No. 14/096,269 is a continuation of application No. PCT/EP2012/060720, filed on Jun. 6, 2012.

(30) Foreign Application Priority Data
Jun. 10, 2011 (DE) 10 2011 077 364.9

(51) Int. Cl. A61K 8/81; A61Q 5/06

(52) U.S. Cl. CPC A61K 8/8152; A61K 8/8176; A61Q 5/06; A61K 2800/594

(57) ABSTRACT

Agents for the temporary shaping of keratinic fibers include in a cosmetically acceptable carrier—relative in each case to the weight thereof—1 to 20 wt. % of at least one crosslinked, amphiphilic, anionic polymer comprising at least one structural unit of formula (I) as described herein and at least one structural unit of formula (II) as described herein, and 1 to 20 wt. % of at least one uncrosslinked, anionic polymer comprising at least one structural unit of formula (III) as described herein and at least one structural unit of formula (IV) as described herein, and 1 to 40 wt. % of at least one fixing non-ionic polymer from the group comprising (c1) non-ionic polymers having at least one structural element of formula (N5) as described herein, (c2) homopolymers and non-ionic copolymers of N-vinylpyrrolidone, and (c3) non-ionic copolymers of isobutene.

### CROSS-REFERENCE TO RELATED APPLICATION

This application is a continuation of U.S. application Ser. No. 14/096,269, filed on Dec. 4, 2013 wherein such application is hereby incorporated in its entirety, by reference.

### FIELD OF THE INVENTION

The present invention generally relates to agents for hair treatment, containing a combination of at least one special crosslinked amphiphilic, anionic polymer and at least one further special uncrosslinked amphiphilic, anionic polymer and at least one third polymer, the use of said agents for the temporary shaping and/or for the care of keratin-containing fibers, and hair gels based on said agents.

### BACKGROUND OF THE INVENTION

Keratin-containing fibers are understood in principle to be all animal hair, for example wool, horsehair, angora hair, fur, feathers and products or textiles manufactured therefrom. The keratinic fibers are however preferably human hair.

An attractive-looking hairstyle is today regarded as an essential element of a well-groomed exterior. The latest fashion trends mean that for many hair types, hairstyles that are regarded as fashionable can only be constructed or maintained for an extended period of up to several days using active fixing agents. Therefore hair treatment agents that provide a permanent or temporary shaping of the hair have an important role to play. Temporary shaping effects which offer good hold without adversely affecting the healthy appearance

Corresponding agents for temporary shaping conventionally contain synthetic polymers as the shaping component. Preparations containing a dissolved or dispersed polymer can be applied to the hair by means of propellants or via a pump mechanism. Hair gels and hair waxes in particular however are generally not applied directly to the hair but are distributed in the hair using a comb or the hands.

The most important property of an agent for temporarily shaping keratinic fibers, also referred to below as a styling agent, consists in giving the treated fibers in the created shape the strongest possible hold. If the keratinic fibers are human hair, this property is also described as a strong styling hold or a high degree of hold of the styling agent. The styling hold is substantially determined by the nature and amount of the synthetic polymer that is used, although the further constituents of the styling agent can also have an influence.

Products having high degrees of hold always involve the risk of visually unattractive product residues in the hair (flaking); in addition, when the degree of hold is increased by raising the polymer content a plateau is generally reached, above which it is difficult to increase the degree of hold by adding further polymer.

The object of the present invention was therefore to provide an agent for temporarily shaping keratinic fibers that is distinguished by a high to maximum degree of hold and has no flaking problems. In addition, it should be possible to achieve the degrees of hold with moderate amounts of polymer, and the degrees of hold should be achieved immediately after application without the need for long drying times.

Surprisingly it has now been found that this can be achieved through the combination of polymers according to the invention.

Furthermore, other desirable features and characteristics of the present invention will become apparent from the subsequent detailed description of the invention and the appended claims, taken in conjunction with the accompanying drawings and this background of the invention.

### BRIEF SUMMARY OF THE INVENTION

An agent for treating keratin-containing fibers, in particular human hair, containing in a cosmetically acceptable carrier—relative in each case to the weight of the agent—

(a) 1 to 20 wt. % of at least one crosslinked, amphiphilic, anionic polymer comprising at least one structural unit of formula (I) and at least one structural unit of formula (II),

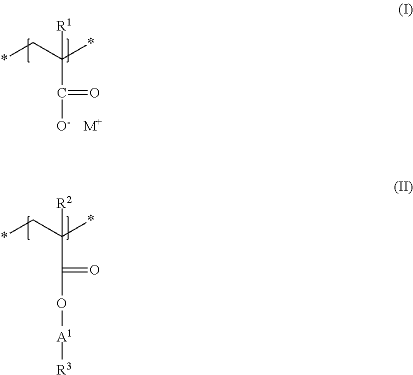

in which
R¹ and R² independently of one another denote a hydrogen atom or a methyl group,
R³ denotes a (C₈ to C₃₀) alkyl group,
M⁺ denotes a physiologically acceptable cation and
A¹ denotes a *—(CH₂CH₂O)ₓ—* group in which x denotes an integer from 5 to 35, a *—(CH₂CHMeO)_{y}—* group in which y denotes an integer from 5 to 35 or a *—(CH₂CH₂O)ₓ—(CH₂CHMeO)_{y}—* group in which the sum of x+y denotes an integer from 5 to 35 and x and y are greater than zero
and
(b) 1 to 20 wt. % of at least one uncrosslinked, anionic polymer comprising at least one structural unit of formula (III) and at least one structural unit of formula (IV),

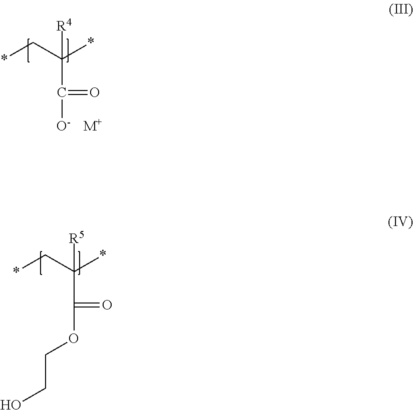

in which
R⁴ and R⁵ independently of one another denote a hydrogen atom or a methyl group,
M⁺ denotes a physiologically acceptable cation,
(c) 1 to 40 wt. % of at least one fixing non-ionic polymer from the group comprising
(c1) non-ionic polymers having at least one structural element of formula (N5)

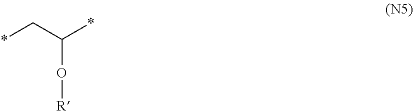

in which R′ denotes a hydrogen atom, an alkyl group or an alkanoyl group, preferably an acetyl group,

(c2) homopolymers and non-ionic copolymers of N-vinylpyrrolidone,
(c3) non-ionic copolymers of isobutene.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

In a first embodiment the present invention provides agents for treating keratin-containing fibers, in particular human hair, containing in a cosmetically acceptable carrier—relative in each case to the weight of the agent—

(a) 1 to 20 wt. % of at least one crosslinked, amphiphilic, anionic polymer comprising at least one structural unit of formula (I) and at least one structural unit of formula (II),

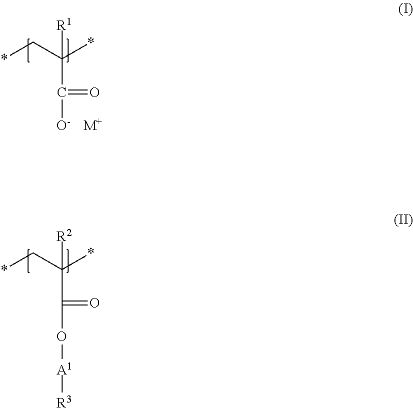

in which
R¹ and R² independently of one another denote a hydrogen atom or a methyl group,
R³ denotes a (C₈ to C₃₀) alkyl group,
M⁺ denotes a physiologically acceptable cation and
A¹ denotes a *—(CH₂CH₂O)ₓ—* group in which x denotes an integer from 5 to 35, a *—(CH₂CHMeO)_{y}—* group in which y denotes an integer from 5 to 35 or a *—(CH₂CH₂O)ₓ—(CH₂CHMeO)_{y}—* group in which the sum of x+y denotes an integer from 5 to 35 and x and y are greater than zero, and
(b) 1 to 20 wt. % of at least one uncrosslinked, anionic polymer comprising at least one structural unit of formula (III) and at least one structural unit of formula (IV),

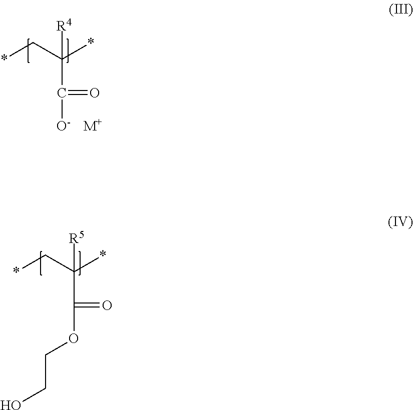

in which
R⁴ and R⁵ independently of one another denote a hydrogen atom or a methyl group,
M⁺ denotes a physiologically acceptable cation,
(c) 1 to 40 wt. % of at least one fixing non-ionic polymer from the group comprising
(c1) non-ionic polymers having at least one structural element of formula (N5)

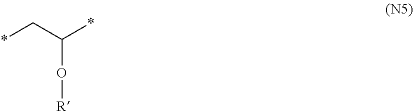

in which R′ denotes a hydrogen atom, an alkyl group or an alkanoyl group, preferably an acetyl group,
(c2) homopolymers and non-ionic copolymers of N-vinylpyrrolidone,
(c3) non-ionic copolymers of isobutene.

Examples of (C₁ to C₄) alkyl groups according to the invention are methyl, ethyl, propyl, isopropyl, butyl, sec-butyl, isobutyl, tert-butyl. Examples of (C₈ to C₃₀) alkyl groups according to the invention are octyl (capryl), decyl (caprinyl), dodecyl (lauryl), tetradecyl (myristyl), hexadecyl (cetyl), octadecyl (stearyl), eicosyl (arachyl), docosyl (behenyl).

In the above formulae and all subsequent formulae, a chemical bond marked with the symbol * denotes a free valence of the corresponding structural fragment.

In particular, metal cations of the physiologically acceptable metals from groups Ia, Ib, IIa, IIb, IIIb, VIa or VIII of the periodic table, ammonium ions and cationic organic compounds having a quaternized nitrogen atom are suitable as physiologically acceptable cations M⁺ to offset the negative charge of the amphiphilic, anionic polymers. The latter are formed for example by protonating primary, secondary or tertiary organic amines with an acid or by permanent quaternization of said organic amines. Examples of these cationic organic ammonium compounds are 2-ammonioethanol and 2-trimethyl ammonioethanol.

Within the meaning of the invention “crosslinked” or “crosslinking” is understood to mean the linking together of polymer chains by chemical bonding to form a network. This covalent linking of the polymer chains can take place by means of direct covalent bonding or can be mediated by means a molecule fragment bridging the polymer chains. The molecule fragment binds in each case to the polymer chains that are bridged by the molecule fragment by means of covalent chemical bonding. Within the meaning of invention “uncrosslinked” is understood to mean that there is no “crosslinking” as defined above.

The person skilled in the art generally understands “amphiphilic” to mean that a single molecule encompasses both hydrophilic structural elements (for example those of formulae (I) or (III)) and lipophilic structural elements (for example those of formulae (II) or (IV)).

Agents that are preferred according to the invention have the characterizing feature that according to formula (II) A¹ denotes a *—(CH₂CH₂O)ₓ—* group in which x denotes an integer from 5 to 35, in particular an integer from 10 to 24.

Agents that are further preferred according to the invention have the characterizing feature that according to formula (II) R² preferably denotes a methyl group.

Crosslinking of the crosslinked, amphiphilic, anionic polymers (a) can preferably be accomplished through the use of at least one crosslinking monomer. It is in turn preferable to choose the crosslinking monomers from at least one compound of the group formed from polyunsaturated aromatic monomers (such as for example divinylbenzene, divinylnaphthalene, trivinylbenzene), polyunsaturated alicyclic monomers (such as for example 1,2,4-trivinylcyclohexane), difunctional esters of phthalic acid (such as for example diallylphthalate), polyunsaturated aliphatic monomers (such as for example dienes, trienes, tetraenes such as isoprene, 1,3-butadiene, 1,5-hexadiene, 1,5,9-decatriene, 1,9-decadiene, 1,5-heptadiene), polyalkenyl ethers (such as for example triallyl pentaerythritol, diallyl pentaerythritol, diallyl sucrose, octaallyl sucrose, trimethylolpropane diallyl ether), polyunsaturated esters of polyalcohols or polyacids (such as for example 1,6-hexanediol di(meth)acrylate, tetramethylene tri(meth)acrylate, allyl acrylate, diallyl itaconate, diallyl fumarate, diallyl maleate, trimethylolpropane tri(meth)acrylate, trimethylolpropane di(meth)acrylate, polyethyleneglycol di(meth)acrylate), alkylene bisacrylamides (such as for example methylene bisacrylamide, propylene bisacrylamide), hydroxy and carboxy derivatives of methylene bisacrylamide (such as for example N,N′-bismethylol methylene bisacrylamide), polyethyleneglycol di(meth)acrylates (such as for example ethyleneglycol di(meth)acrylate, diethyleneglycol di(meth)acrylate, triethyleneglycol di(meth)acrylate), polyunsaturated silanes (such as for example dimethyldivinylsilane, methyltrivinylsilane, allyldimethylvinylsilane, diallyldimethylsilane, tetravinylsilane), N-methylolacrylamide; N-alkoxy(meth)acrylamide, wherein the alkoxy group is a (C₁ to C₁₈) alkoxy group, unsaturated hydrolyzable silanes (such as for example triethoxyvinylsilane, trisisopropoxyvinylsilane, 3-triethoxysilyl propyl methacrylate), hydrolyzable silanes (such as for example ethyltriethoxysilane, ethyltrimethoxysilane), epoxy-substituted hydrolyzable silanes (such as for example 2-(3,4-epoxycyclohexyl)ethyltriethoxysilane, 3-glycidoxypropyltrimethoxysilane), polyisocyanates (such as for example 1,4-diisocyanatobutane, 1,6-diisocyanatohexane, 1,4-phenylene diisocyanate, 4,4′-oxybis(phenyl isocyanate), unsaturated epoxides (such as for example glycidyl methacrylates, allyl glycidyl ethers), polyepoxides (such as for example diglycidyl ethers, 1,2,5,6-diepoxyhexane, ethyleneglycol diglycidyl ethers), ethoxylated polyols (such as for example diols, triols and diphenols, ethoxylated in each case with 2 to 100 mol of ethylene oxide per mol of hydroxyl groups and terminated with a polymerizable unsaturated group, such as for example vinyl ethers, allyl ethers, acrylate esters, methacrylate esters; examples encompass bisphenol A ethoxylated di(meth)acrylate, bisphenol F ethoxylated di(meth)acrylate, ethoxylated trimethylolpropane tri(meth)acrylates, acrylate and methacrylate esters of polyols having at least two acrylate ester or methacrylate ester functionalities (such as for example trimethylolpropane triacrylate (TMPTA), trimethylolpropane ethoxylated (15) triacrylate (TMPEO15TA), trimethylolpropane dimethacrylate, triethyleneglycol dimethacrylate (TEGDMA), bisphenol A dimethacrylate ethoxylated with 30 mol of ethylene oxide (EOBDMA)).

The crosslinked, amphiphilic, anionic polymers (a) are preferably included in the agents according to the invention in an amount from 0.1 wt. % to 5.0 wt. % particularly preferably from 0.2 wt. % to 2 wt. %, most particularly preferably from 0.2 wt. % to 1.5 wt. %, relative in each case to the weight of the total agent.

Particularly preferred crosslinked amphiphilic anionic polymers (a) according to the invention additionally contain at least one structural unit of formula (V)

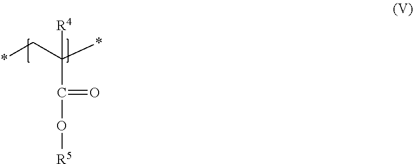

in which
R⁴ denotes a hydrogen atom or a methyl group,
R⁵ denotes a (C₂ to C₄) alkyl group, in particular ethyl.

It is preferable according to the invention to select as the crosslinked, amphiphilic, anionic polymer (a) at least one polymer comprising at least one structural unit of formula (I) and at least one structural unit of formula (II-a),

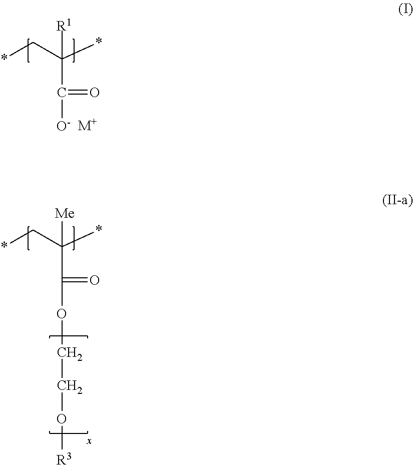

in which
R¹ denotes a hydrogen atom or a methyl group,
R³ denotes a (C₈ to C₃₀) alkyl group,
M⁺ denotes a physiologically acceptable cation and
x denotes an integer from 5 to 35, in particular an integer from 10 to 24.

It is moreover particularly preferably to select the crosslinked, amphiphilic, anionic polymers (a) from at least one polymer comprising at least one structural unit of formula (I) and at least one structural unit of formula (II-a) and at least one structural unit of formula (V),

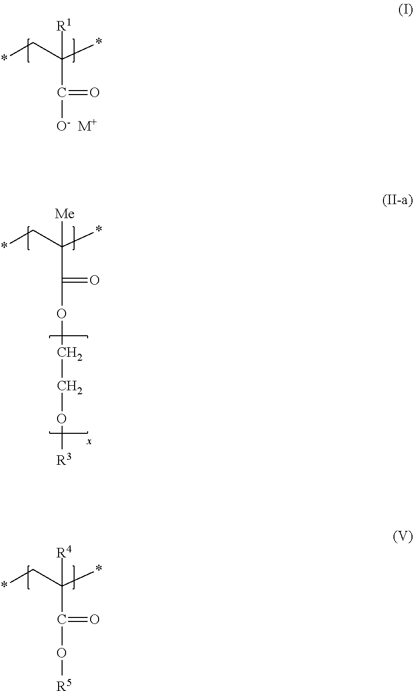

in which
R¹ and R⁴ independently of one another denote a hydrogen atom or a methyl group,
R³ denotes a (C₈ to C₃₀) alkyl group,
R⁵ denotes a (C₂ to C₄) alkyl group, in particular ethyl,
M⁺ denotes a physiologically acceptable cation and
x denotes an integer from 5 to 35, in particular an integer from 10 to 24.

A most particularly preferred polymer (a) is a crosslinked, amphiphilic, anionic polymer which comes under the MCI name Acrylates/Steareth-20 Methacrylate Crosspolymer. It has 20 units of ethylene oxide (x according to formula (II-a)=20) and is etherified with stearyl alcohol (R³ according to formula (II-a)=stearyl). Such polymers are sold for example under the trade name Aculyn® 88 by Rohm & Haas in the form of a 28 to 30 wt. % dispersion in water.

In addition to the crosslinked amphiphilic, anionic polymers defined above, the agent according to the invention also mandatorily contains at least one uncrosslinked, amphiphilic, anionic polymer (b) as defined above.

Preferred agents within the meaning of the invention contain the uncrosslinked, amphiphilic, anionic polymers (b) in an amount from 0.1 wt. % to 5.0 wt. % particularly preferably from 0.2 wt. % to 5.0 wt. %, most particularly preferably from 0.1 wt. % to 2.0 wt. %, relative in each case to the weight of the agent.

The crosslinked, amphiphilic, anionic polymers (a) and the uncrosslinked, amphiphilic, anionic polymers (b) are preferably used according to the invention in a weight ratio of [polymer (a) to polymer (b)] from 1 to 5 to 5 to 1, in particular from 1 to 2 to 5 to 1, most particularly preferably from 1 to 1.5 to 2 to 1.

According to formula (IV) A² preferably denotes a *—(CH₂CH₂O)ₓ—* group in which x denotes an integer from 5 to 35, in particular an integer from 15 to 30.

According to formula (IV) R⁵ preferably denotes a hydrogen atom.

Agents that are preferred according to the invention have the characterizing feature that the uncrosslinked, anionic polymer (b) additionally contains at least one structural unit of formula (V),

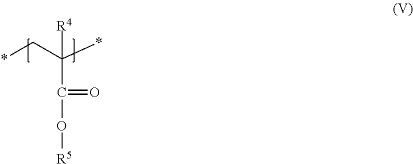

in which
R⁴ denotes a hydrogen atom or a methyl group,
R⁵ denotes a (C₂ to C₄) alkyl group, in particular methyl or n-butyl.

Particularly preferred uncrosslinked amphiphilic anionic polymers (b) according to the invention additionally contain at least one structural unit of formula (V)

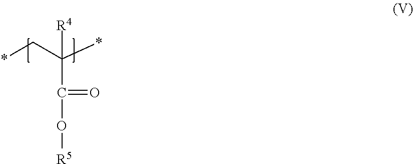

in which
R⁴ denotes a hydrogen atom or a methyl group,
R⁵ denotes a (C₂ to C₄) alkyl group, in particular ethyl.

Most highly preferred agents according to the invention have the characterizing feature that the uncrosslinked, anionic polymer (b) is a linear tetrapolymer consisting of methacrylic acid, hydroxyethyl methacrylate, methyl methacrylate and butyl acrylate.

In addition to the two aforementioned polymers, the agents according to the invention contain as a third polymer 1 to 40 wt. % of at least one fixing, non-ionic polymer from the group comprising

(c1) non-ionic polymers having at least one structural element of formula (N5)

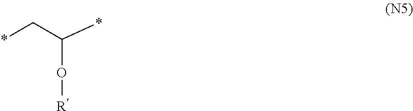

in which R′ denotes a hydrogen atom, an alkyl group or an alkanoyl group, preferably an acetyl group,
(c2) homopolymers and non-ionic copolymers of N-vinylpyrrolidone,
(c3) non-ionic copolymers of isobutene.

Non-ionic polymers based on ethylenically unsaturated monomers that are preferably suitable as the additional film-forming and/or fixing polymer are non-ionic polymers containing at least one of the following structural units:

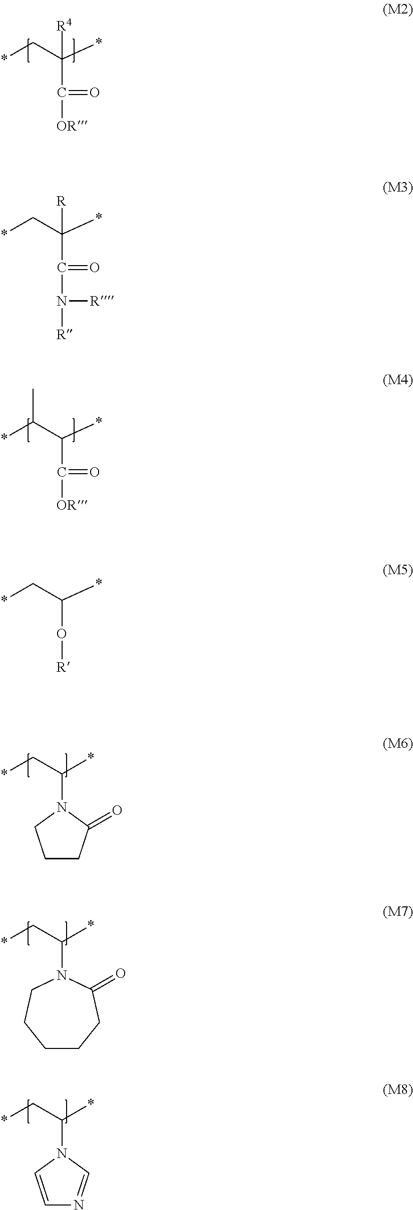

in which
R denotes a hydrogen atom or a methyl group,
R′ denotes a hydrogen atom or a (C₁ to C₄) acyl group,
R″ and R″″ independently of one another denote a (C₁ to C₇) alkyl group or a hydrogen atom
R′″ denotes a linear or branched (C₁ to C₄) alkyl group or a (C₂ to C₄) hydroxyalkyl group.

Preferred non-ionic film-forming and/or non-ionic hair-fixing polymers are homopolymers or copolymers constructed from at least one of the following monomers: vinylpyrrolidone, vinylcaprolactam, vinyl esters such as for example vinyl acetate, vinyl alcohol, acrylamide, methacrylamide, alkyl and dialkyl acrylamide, alkyl and dialkyl methacrylamide, alkyl acrylate, alkyl methacrylate, the alkyl groups of these monomers preferably being selected in each case from (C₁ to C₃) alkyl groups.

Non-ionic polymers based on ethylenically unsaturated monomers that are particularly suitable for the agents according to the invention contain at least one of the following structural units:

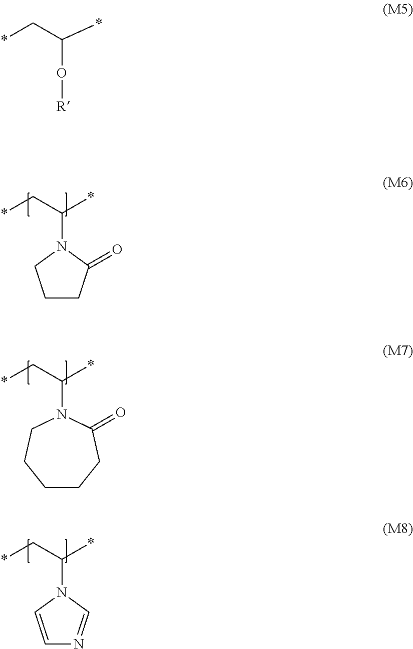

in which
R′ denotes a hydrogen atom or a (C₁ to C₃₀) acyl group, in particular a hydrogen atom or an acetyl group.

Homopolymers of vinylcaprolactam or of vinylpyrrolidone (such as for example Luviskol® K 90 or Luviskol® K 85 from BASF SE), copolymers of vinylpyrrolidone and vinyl acetate (such as are sold for example under the trademarks Luviskol® VA 37, Luviskol® VA 55, Luviskol® VA 64 and Luviskol® VA 73 by BASF SE), terpolymers of vinylpyrrolidone, vinyl acetate and vinylpropionate, polyacrylamides (such as for example Akypomine® P 191 from CHEM-Y), polyvinyl alcohols (which are sold for example under the trade names Elvanol® by Du Pont or Vinol® 523/540 by Air Products), terpolymers of vinylpyrrolidone, methacrylamide and vinylimidazole (such as for example Luviset® Clear from BASF SE) are suitable in particular.

In addition to the non-ionic polymers based on ethylenically unsaturated monomers, non-ionic cellulose derivatives, which are preferably selected from methyl cellulose and in particular from cellulose ethers, such as hydroxypropyl celluloses (e.g. hydroxypropyl cellulose with a molecular weight from 30,000 to 50,000 g/mol, which is sold for example under the trade name Nisso S1® by Lehmann & Voss, Hamburg), hydroxyethyl celluloses, such as are sold for example under the trademarks Culminal® and Benecel® (AQUALON) and Natrosol® types (Hercules), are also suitable as film-forming and/or fixing polymers for the preferred embodiment of the technical teaching.

In summary, agents according to the invention are preferred which as the fixing, non-ionic polymer (c) contain at least one polymer from the group comprising

polyvinylpyrrolidone,

copolymers of N-vinylpyrrolidone and vinyl esters of carboxylic acids having 2 to 18 carbon atoms, in particular of N-vinylpyrrolidone and vinyl acetate,

copolymers of N-vinylpyrrolidone and N-vinylimidazole and methacrylamide,

copolymers of N-vinylpyrrolidone and N-vinylimidazole and acrylamide,

copolymers of N-vinylpyrrolidone with N,N-di(C₁ to C₄) alkylamino-(C₂ to C₄)-alkylacrylamide,

copolymers of N-vinylpyrrolidone with N,N-di(C₁ to C₄) alkylamino-(C₂ to C₄)-alkylacrylamide.

Irrespective of the polymer(s) used in each case, the polymers are preferably used within relatively narrow quantity ranges. Through the combination according to the invention, maximum degrees of hold are achievable with even small amounts of polymer. Agents according to the invention are preferred which contain—relative to their weight—

the crosslinked, amphiphilic, anionic polymers (a) in an amount from 1.5 to 17.5 wt. %, preferably from 2 to 15 wt. %, more preferably from 3 to 12.5 wt. % and in particular from 5 to 10 wt. %,

the uncrosslinked, anionic polymers (b) in an amount from 1.5 to 17.5 wt. %, preferably from 2 to 15 wt. %, more preferably from 2.5 to 12.5 wt. % and in particular from 3 to 10 wt. %,

the fixing non-ionic polymers (c) in an amount from 1.5 to 35 wt. %, preferably from 2 to 30 wt. %, more preferably from 3 to 25 wt. % and in particular from 5 to 20 wt. %.

For the achievement of maximum degrees of hold and for the minimizing of flaking phenomena it has proved most particularly preferable for the polymers (a) and (b) to be used in specific proportions to one another. Agents according to the invention are preferred in which the crosslinked, amphiphilic, anionic polymers (a) and the uncrosslinked, anionic polymers (b) are used in a weight ratio from 1 to 5 to 5 to 1, in particular from 1 to 2 to 5 to 1, particularly preferably from 1 to 1.5 to 2 to 1.

In addition to the polymers, the agents according to the invention can contain at least one wax having a melting point in a range from 40° C. to 90° C.

Waxes are generally of a solid to crumbly hard consistency, coarsely to finely crystalline, translucent to opaque, but not glassy, and melt above 40° C. without decomposing. At even a little above the melting point they are of low viscosity and have a highly temperature-dependent consistency and solubility. Natural vegetable waxes, for example candelilla wax, carnauba wax, Japan wax, sugar cane wax, ouricury wax, cork wax, sunflower wax, fruit waxes such as orange wax, lemon wax, grapefruit wax, and animal waxes, for example beeswax, shellac wax and spermaceti wax, are preferred for example according to the invention. Within the meaning of the invention it can be particularly preferable to use hydrogenated or hardened waxes. Chemically modified waxes, in particular hard waxes such as for example montan ester waxes, hydrogenated jojoba waxes and Sasol waxes, can also be used as the wax component. Synthetic waxes that are likewise preferred according to the invention include for example polyalkylene waxes, in particular polyethylene waxes, and polyethylene glycol waxes, C₂₀-C₄₀ dialkyl esters of dimeric acids, C₃₀₋₅₀ alkyl beeswax and alkyl and alkylaryl esters of dimeric fatty acids.

A particularly preferred wax component is selected from at least one ester of a saturated, monohydric C₁₆-C₅₀ alcohol and a saturated C₈-C₃₆ monocarboxylic acid. According to the invention these include lactides, the cyclic double esters of alpha-hydroxycarboxylic acids of the corresponding chain length. Esters of fatty acids and long-chain alcohols have proved particularly advantageous for the compositions that are preferred according to the invention. The esters consist of saturated, branched or unbranched monocarboxylic acids and saturated, branched or unbranched monohydric alcohols. Esters of aromatic carboxylic acids or hydroxycarboxylic acids (e.g. 12-hydroxystearic acid) and saturated, branched or unbranched alcohols can also be used according to the invention, provided that the wax component has a melting point >50° C. It is particularly preferable to select the wax components from the group of esters of saturated, branched or unbranched alkane carboxylic acids having a chain length of 12 to 24 C atoms and saturated, branched or unbranched alcohols having a chain length of 16 to 50 C atoms that have a melting point of >50° C.

In particular, C₁₆₋₃₆ alkyl stearates and C₁₈₋₃₈ alkyl hydroxystearoyl stearates, C₂₀₋₄₀ alkyl erucates and cetearyl behenate can be preferred as the wax component. The wax or wax components have a melting point >50° C., preferably >60° C.

A particularly preferred embodiment of the invention contains a C₂₀-C₄₀ alkyl stearate as the wax component. This ester is known under the name Kesterwachs® K82H or Kesterwachs® K80H and is sold by Koster Keunen Inc. It is the synthetic simulation of the monoester fraction of beeswax and is distinguished by its hardness, its oil gelling capacity and its broad compatibility with lipid components. A further particularly preferred embodiment of the invention contains cetearyl behenate, i.e. mixtures of cetyl behenate and stearyl behenate, as the wax component. This ester is known under the name Kesterwachs® K62 and is sold by Koster Keunen Inc.

Other preferred wax components having a melting point >50° C. are the triglycerides of saturated and optionally hydroxylated C₁₂₋₃₀ fatty acids, such as hardened triglyceride fats (hydrogenated palm oil, hydrogenated coconut oil, hydrogenated castor oil), glyceryl tribehenate (tribehenin) or glyceryl tri-12-hydroxystearate, also synthetic full esters of fatty acids and glycols or polyols having 2 to 6 carbon atoms, provided they have a melting point above 50° C., for example preferably C₁₈-C₃₈ acid triglyceride (Syncrowax® HGL-C). Hydrogenated castor oil, obtainable for example as the commercial product Cutina® HR, is particularly preferred according to the invention as the wax component.

Other preferred lipid or wax components having a melting point >50° C. are the saturated linear C₁₄-C₃₆ carboxylic acids, in particular myristic acid, palmitic acid, stearic acid and behenic acid, as well as mixtures of these compounds, for example Syncrowax® AW IC (C₁₈-C₃₆ fatty acids) or Cutina® FS 45 (palmitic and stearic acid).

Further preferred lipid or wax components having a melting point in the range from 30 to 150° C. are linear, saturated C₈₋₃₀ fatty acids. Linear, saturated C₁₀₋₂₂ fatty acids are preferred. Preferred fatty acids are hexanoic acid, octanoic acid, 2-ethylhexanoic acid, decanoic acid, lauric acid, isotridecanoic acid, myristic acid, palmitic acid, palmitoleic acid, stearic acid, isostearic acid, oleic acid, elaidic acid, petroselic acid, elaeostearic acid, eicosanoic acid, gadoleic acid, behenic acid and erucic acid and technical mixtures thereof. The use of stearic acid is particularly preferred. The fatty acids that are used can bear one or more hydroxyl groups. Preferred examples are α-hydroxy-C₈-C₁₈-carboxylic acids and 12-hydroxystearic acid.

Further preferred lipid or wax components having a melting point in the range from 30 to 150° C. are fatty alcohols. Saturated, unbranched fatty alcohols having 6-30, preferably 10-22 and most particularly preferably 12-22 carbon atoms can be used as fatty alcohols. Decanol, octanol, erucic alcohol, ricinol alcohol, 12-hydroxystearyl alcohol, stearyl alcohol, cetyl alcohol, lauryl alcohol, myristyl alcohol, arachidyl alcohol, capryl alcohol, caprinic alcohol and behenyl alcohol, for example, can be used within the meaning of the invention.

Preferred compositions according to the invention have the characterizing feature that the wax component is selected from esters of a saturated, monohydric C₁₆-C₆₀ alkanol and a saturated C₆-C₃₆ monocarboxylic acid, in particular cetyl behenate, stearyl behenate and C₂₀-C₄₀ alkyl stearate, glycerol triesters of saturated linear C₁₂-C₃₀ carboxylic acids, which can be hydroxylated, candelilla wax, carnauba wax, beeswax, saturated linear C₁₄-C₃₆ carboxylic acids and mixtures of the aforementioned substances. Particularly preferred wax component mixtures are selected from mixtures of cetyl behenate, stearyl behenate, hydrogenated castor oil, palmitic acid and stearic acid. Further particularly preferred wax component mixtures are selected from mixtures of C₂₀-C₄₀ alkyl stearate, hydrogenated castor oil, palmitic acid and stearic acid.

Particularly preferred compositions according to the invention have the characterizing feature that the wax component is selected from mixtures of esters of a saturated, monohydric C₁₆-C₆₀ alkanol and a saturated C₈-C₃₆ monocarboxylic acid, in particular C₂₀-C₄₀ alkyl stearate, glycerol triesters of saturated linear C₁₂-C₃₀ carboxylic acids, which can be hydroxylated, in particular hydrogenated castor oil, and saturated linear C₁₄-C₃₆ carboxylic acids, in particular palmitic acid and stearic acid.

The agents according to the invention contain the ingredients in a cosmetically acceptable carrier. Preferred cosmetically acceptable carriers are aqueous, alcoholic or aqueous-alcoholic media having preferably at least 10 wt. % water, relative to the total agent. The low alcohols having 1 to 4 carbon atoms that are conventionally used for cosmetic purposes, such as for example ethanol and isopropanol, can be included in particular as alcohols.

Organic solvents or a mixture of solvents with a boiling point below 400° C. can be included as additional co-solvents in an amount from 0.1 to 15 percent by weight, preferably from 1 to 10 percent by weight, relative to the total agent. Unbranched or branched hydrocarbons, such as pentane, hexane, isopentane, and cyclic hydrocarbons, such as cyclopentane and cyclohexane, are particularly suitable as additional co-solvents. Other particularly preferred water-soluble solvents are glycerol, ethylene glycol, butylene glycol and propylene glycol in an amount of up to 30 wt. % relative to the total agent.

Most particularly preferred agents according to the invention have a high water content. It has been found that the adjustment of shine, remodeling ability and degree of hold in the compositions according to the invention is particularly successful if they contain high proportions of water. Particularly preferred agents according to the invention therefore have the characterizing feature that they contain, relative to their weight, 40 to 95 wt. %, preferably 45 to 92.5 wt. %, more preferably 50 to 90 wt. %, still more preferably 55 to 87.5 wt. % and in particular 60 to 85 wt. % of water.

The applicability of the compositions according to the invention can be further increased through the use of small amounts of one or more polyhydric alcohols. Preferred agents according to the invention contain, relative to their weight, 0.25 to 5 wt. %, preferably 0.5 to 4 wt. %, more preferably 0.75 to 3 wt. % and in particular 1 to 2.5 wt. % of at least one polyhydric alcohol form the group comprising glycerol and/or propanediol-1,2.

The agents preferably have a pH of 2 to 11. The pH range between 4 and 9 is particularly preferred. Unless otherwise specified, within the meaning of this document the pH values stated relate to the pH at 25° C.

The agents according to the invention can furthermore contain the auxiliary substances and additives that are conventionally added to the various cosmetic agents.

Suitable auxiliary substances and additives include in particular care substances. These are used in both skin and hair treatment agents and with an appropriate choice of the care substance can be incorporated into creams, shampoos, hair rinses, hair masks, gels, pump and aerosol sprays and foam products, for example.

An agent according to the invention can for example contain at least one protein hydrolysate and/or a derivative thereof as a care substance.

Protein hydrolysates are mixtures of products which are obtained by acidically, basically or enzymatically catalyzed breakdown of proteins. The molecular weight of the protein hydrolysates for use according to the invention is between 75, the molecular weight for glycine, and 200,000; the molecular weight is preferably 75 to 50,000 and most particularly preferably 75 to 20,000 daltons.

According to the invention protein hydrolysates of both plant and animal or marine or synthetic origin can be used. Animal protein hydrolysates are for example elastin, collagen, keratin, silk and milk protein hydrolysates, which can also be present in the form of salts. Such products are sold for example under the trademarks Dehylan® (Cognis), Promois® (Interorgana), Collapuron® (Cognis), Nutrilan® (Cognis), Gelita-Sol® (Deutsche Gelatine Fabriken Stoess & Co), Lexein® (Inolex), Sericin (Pentapharm) and Kerasol® (Croda). The use of silk protein hydrolysates is of particular interest. The protein hydrolysates are contained in the agents according to the invention in concentrations for example from 0.01 wt. % to 20 wt. %, preferably from 0.05 wt. % to 15 wt. % and most particularly preferably in amounts from 0.05 wt. % to 5 wt. %, relative in each case to the total application preparation.

Cationic surfactants are moreover suitable as a care substance from a different substance class. Cationic surfactants of the quaternary ammonium compound, esterquat and amidoamine type are preferred according to the invention. Preferred quaternary ammonium compounds are ammonium halides, in particular chlorides and bromides, such as alkyltrimethylammonium chlorides, dialkyldimethylammonium chlorides and trialkylmethylammonium chlorides, for example cetyltrimethylammonium chloride, stearyltrimethylammonium chloride, distearyldimethylammonium chloride, lauryldimethylammonium chloride, lauryldimethylbenzylammonium chloride and tricetylmethylammonium chloride, as well as the imidazolium compounds known under the INCI names Quaternium-27 and Quaternium-83. The long alkyl chains of the aforementioned surfactants preferably have 10 to 18 carbon atoms. Esterquats are known substances containing both at least one ester function and at least one quaternary ammonium group as a structural element. Preferred esterquats are quaternized ester salts of fatty acids with triethanolamine, quaternized ester salts of fatty acids with diethanol alkyl amines and quaternized ester salts of fatty acids with 1,2-dihydroxypropyl dialkylamines. Such products are sold under the trademarks Stepantex®, Dehyquart® and Armocare®, for example. The products Armocare® VGH-70, an N,N-bis(2-palmitoyloxyethyl)dimethylammonium chloride, as well as Dehyquart® F-75, Dehyquart® C-4046, Dehyquart® L80 and Dehyquart® AU-35 are examples of such esterquats. The alkylamidoamines are conventionally produced by amidation of natural or synthetic fatty acids and fatty acid cuts with dialkyl amino amines. A compound from this group of substances that is particularly suitable according to the invention is the stearamidopropyl dimethylamine which is commercially available under the name Tegoamid® S 18.

The cationic surfactants are preferably contained in the agents according to the invention in amounts from 0.05 to 10 wt. %, relative to the total application composition. Amounts from 0.1 to 5 wt. % are particularly preferred.

Conditioning polymers are likewise suitable as a care substance. It should be mentioned at this point that some conditioning polymers also have film-forming and/or fixing properties and can therefore also be included in the list of suitable film-forming and/or fixing polymers.

A first group of conditioning polymers are the cationic polymers. Cationic polymers are understood to be polymers having a group in the main and/or side chain which can be “temporarily” or “permanently” cationic. Polymers which have a cationic group irrespective of the pH of the agent are described as “permanently cationic” according to the invention. These are generally polymers containing a quaternary nitrogen atom, in the form of an ammonium group for example. Preferred cationic groups are quaternary ammonium groups. In particular, polymers in which the quaternary ammonium group is bonded via a C₁₋₄ hydrocarbon group to a polymer main chain synthesized from acrylic acid, methacrylic acid or derivatives thereof have proved to be particularly suitable. Homopolymers of the general formula (G1-I),

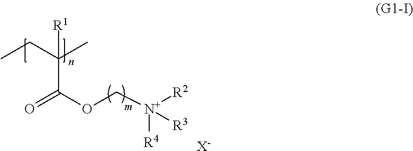

in which R¹ is —H or —CH₃, R², R³ and R⁴ are independently of one another selected from C₁₋₄ alkyl, alkenyl or hydroxyalkyl groups, m=1, 2, 3 or 4, n is a natural number and X⁻ is a physiologically acceptable organic or inorganic anion, as well as copolymers consisting substantially of the monomer units shown in formula (G1-I) along with non-ionogenic monomer units, are particularly preferred cationic polymers. In the context of these polymers those for which at least one of the following conditions applies are preferred according to the invention: R¹ denotes a methyl group, R², R³ and R⁴ denote methyl groups, m has the value 2.

Suitable physiologically acceptable counterions X⁻ are for example halide ions, sulfate ions, phosphate ions, methosulfate ions as well as organic ions such as lactate, citrate, tartrate and acetate ions. Halide ions, in particular chloride, are preferred.

A particularly suitable homopolymer is the optionally crosslinked poly(methacryloyloxyethyl trimethylammonium chloride) with the INCI name Polyquaternium-37. Crosslinking can take place if desired with the aid of polyolefinically unsaturated compounds, for example divinyl benzene, tetraallyl oxyethane, methylene bisacrylamide, diallyl ether, polyallyl polyglyceryl ether, or allyl ethers of sugars or sugar derivatives such as erythritol, pentaerythritol, arabitol, mannitol, sorbitol, sucrose or glucose. Methylene bisacrylamide is a preferred crosslinking agent.

The homopolymer is preferably used in the form of a non-aqueous polymer dispersion which should have a polymer content of not less than 30 wt. %. Such polymer dispersions are commercially available under the names Salcare® SC 95 (approx. 50% polymer content, other components: mineral oil (INCI name: Mineral Oil) and tridecyl polyoxypropylene polyoxyethylene ether (INCI name: PPG-1-Trideceth-6)) and Salcare® SC 96 (approx. 50% polymer content, other components: mixture of diesters of propylene glycol with a mixture of caprylic and capric acid (INCI name: Propylene Glycol Dicaprylate/Dicaprate) and tridecyl polyoxypropylene polyoxyethylene ether (INCI name: PPG-1-Trideceth-6)).

Copolymers with monomer units according to formula (G1-I) preferably contain as non-ionogenic monomer units acrylamide, methacrylamide, acrylic acid C₁₋₄ alkyl esters and methacrylic acid C₁₋₄ alkyl esters. Of these non-ionogenic monomers acrylamide is particularly preferred. As in the case of the homopolymers described above, these copolymers too can be crosslinked. A copolymer that is preferred according to the invention is the crosslinked acrylamide-methacryloyloxyethyl trimethylammonium chloride copolymer. Such copolymers, in which the monomers are present in a weight ratio of about 20:80, are available commercially as an approx. 50% non-aqueous polymer dispersion under the name Salcare® SC 92.

Further preferred cationic polymers are for example

quaternized cellulose derivatives such as are available commercially under the names Celquat® and Polymer JR®. The compounds Celquat® H 100, Celquat® L 200 and Polymer JR®400 are preferred quaternized cellulose derivatives,

cationic alkyl polyglycosides according to DE-PS 44 13 686,

cationized honey, for example the commercial product Honeyquat® 50,

cationic guar derivatives, such as in particular the products sold under the trade names Cosmedia®Guar and Jaguar®,

polysiloxanes containing quaternary groups, such as for example the commercially available products Q2-7224 (manufacturer: Dow Corning; a stabilized trimethylsilyl amodimethicone), Dow Corning® 929 Emulsion (containing a hydroxyl-amino-modified silicone, which is also known as amodimethicone), SM-2059 (manufacturer: General Electric), SLM-55067 (manufacturer: Wacker) as well as Abil®-Quat 3270 and 3272 (manufacturer: Th. Goldschmidt), diquaternary polydimethylsiloxanes, Quaternium-80,

polymeric dimethyl diallyl ammonium salts and copolymers thereof with esters and amides of acrylic acid and methacrylic acid. The products available commercially under the names Merquat® 100 (poly(dimethyl diallyl ammonium chloride)) and Merquat®550 (dimethyl diallyl ammonium chloride acrylamide copolymer) are examples of such cationic polymers,

quaternized polyvinyl alcohol,

and the polymers known under the names Polyquaternium-2, Polyquaternium-17, Polyquaternium-18 and Polyquaternium-27 with quaternary nitrogen atoms in the polymer main chain.

Further cationic polymers that can be used according to the invention are the “temporary cationic” polymers. These polymers conventionally contain an amino group that at certain pH values takes the form of a quaternary ammonium group and is therefore cationic. Chitosan and derivatives thereof, such as are widely available commercially under the trade names Hydagen® CMF, Hydagen® HCMF, Kytamer® PC and Chitolam® NB/101, for example, are preferred.

The agents according to the invention preferably contain the conditioning, cationic and/or amphoteric polymers in an amount from 0.01 to 5 wt. %, in particular in an amount from 0.1 to 2 wt. %, relative in each case to the total application preparation.

Further preferred agents according to the invention have the characterizing feature that they additionally contain care substance(s)—relative to their weight—in amounts from 0.001 to 10 wt. %, preferably 0.005 to 7.5 wt. %, particularly preferably 0.01 to 5 wt. % and in particular 0.05 to 2.5 wt. %, with preferred care substance(s) being selected from the group comprising L-carnitine and/or salts thereof; panthenol and/or pantothenic acid; 2-furanones and/or derivatives thereof (in particular pantolactone); taurine and/or salts thereof; niacinamide; ubiquinone; ectoine; allantoin.

L-Carnitine (IUPAC name (R)-(3-Carboxy-2-hydroxypropyl)-N,N,N-trimethylammonium hydroxide), is a naturally occurring, vitamin-like substance. L-Carnitine derivatives that are preferred according to the invention are selected in particular from acetyl L-carnitine, L-carnitine fumarate, L-carnitine citrate, lauroyl L-carnitine and particularly preferably L-carnitine tartrate. The specified L-carnitine compounds are available for example from Lonza GmbH (Wuppertal, Germany). Preferred agents according to the invention have the characterizing feature that they contain—relative to their weight—0.001 to 10 wt. %, preferably 0.005 to 7.5 wt. %, particularly preferably 0.01 to 5 wt. % and in particular 0.05 to 2.5 wt. % of L-carnitine or L-carnitine derivatives, L-carnitine derivatives being preferably selected from acetyl L-carnitine, L-carnitine fumarate, L-carnitine citrate, lauroyl L-carnitine and in particular L-carnitine tartrate.

Panthenol (IUPAC name: (+)-(R)-2,4-Dihydroxy-N-(3-hydroxypropyl)-3,3-dimethylbutyramide) is converted in the body to pantothenic acid. Pantothenic acid is a vitamin from the group of B vitamins (vitamin B5). Preferred agents according to the invention have the characterizing feature that they contain—relative to their weight—0.01 to 5 wt. %, preferably 0.05 to 2.5 wt. %, particularly preferably 0.1 to 1.5 wt. % and in particular 0.25 to 1 wt. % panthenol ((±)-2,4-dihydroxy-N-(3-hydroxypropyl)-3,3-dimethylbutyramide).

A further care enhancer that can preferably be used, which has activating properties, is taurine. Agents that are preferred according to the invention contain—relative to their weight—0.01 to 15 wt. %, preferably 0.025 to 12.5 wt. %, particularly preferably 0.05 to 10 wt. %, more preferably 0.1 to 7.5 wt. % and in particular 0.5 to 5 wt. % of taurine (2-aminoethanesulfonic acid).

A further preferred group of care enhancers in the agents according to the invention are vitamins, provitamins or vitamin precursors. These are described below:

In summary, agents according to the invention are preferred which contain—relative to their weight—0.1 to 5 wt. %, preferably 0.2 to 4 wt. %, particularly preferably 0.25 to 3.5 wt. %, more preferably 0.5 to 3 wt. % and in particular 0.5 to 2.5 wt. % of vitamins and/or provitamins and/or vitamin precursors, which are preferably assigned to groups A, B, C, E, F and H, wherein preferred agents contain (±)-2,4-dihydroxy-N-(3-hydroxypropyl)-3,3-dimethylbutyramide, provitamin B₅) and/or pantothenic acid (vitamin B₃, vitamin B₅) and/or niacin, niacinamide or nicotinamide (vitamin B₃) and/or L-ascorbic acid (vitamin C) and/or thiamine (vitamin BO and/or riboflavin (vitamin B₂, vitamin G) and/or biotin (vitamin B₇, vitamin H) and/or folic acid (vitamin B₉, vitamin Bₑ or vitamin M) and/or vitamin B₆ and/or vitamin B₁₂.

It has been found that certain quinones are particularly suitable as care enhancers. Particularly preferred agents according to the invention have the characterizing feature that as a care substance they contain—relative to their weight—0.0001 to 1 wt. %, preferably 0.001 to 0.5 wt. % and particularly preferably 0.005 to 0.1 wt. % of at least one ubiquinone and/or at least one ubiquinol and/or at least one derivative of these substances, wherein preferred agents contain an ubiquinone of formula (Ubi)

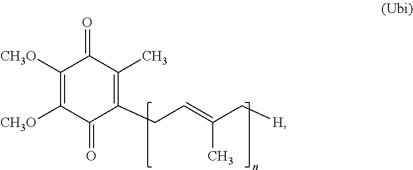

in which n denotes the values 6, 7, 8, 9 or 10, particularly preferably 10 (coenzyme Q10).

As a further care enhancer the agents according to the invention can contain ectoine. Ectoine ((4S)-2-methyl-1,4,5,6-tetrahydropyrimidine-4-carboxylic acid) is a natural substance belonging to the group of compatible solutes.

As a further constituent the agents according to the invention can contain at least one carbohydrate from the group of monosaccharides, disaccharides and/or oligosaccharides. Hair treatment agents that are preferred according to the invention have the characterizing feature that as a care substance they contain—relative to their weight −0.01 to 5 wt. %, preferably 0.05 to 4.5 wt. %, particularly preferably 0.1 to 4 wt. %, more preferably 0.5 to 3.5 wt. % and in particular 0.75 to 2.5 wt. % of carbohydrate(s), selected from monosaccharides, disaccharides and/or oligosaccharides, wherein preferred carbohydrates are selected from

monosaccharides, in particular D-ribose and/or D-xylose and/or L-arabinose and/or D-glucose and/or D-mannose and/or D-galactose and/or D-fructose and/or sorbose and/or L-fucose and/or L-rhamnose

disaccharides, in particular sucrose and/or maltose and/or lactose and/or trehalose and/or cellobiose and/or gentiobiose and/or isomaltose.

In a further preferred embodiment the agents according to the invention can contain emulsifiers (F). Emulsifiers cause water-stable or oil-stable adsorption layers, which protect the dispersed droplets against coalescence and thus stabilize the emulsion, to form at the phase interface. Like surfactants, emulsifiers are therefore made up of a hydrophobic and a hydrophilic molecule part. Hydrophilic emulsifiers preferably form O/W emulsions and hydrophobic emulsifiers preferably form W/O emulsions. An emulsion is understood to be a distribution in droplet form (dispersion) of one liquid in another liquid, expending energy to create stabilizing phase interfaces by means of surfactants. The choice of these emulsifying surfactants or emulsifiers is governed by the substances to be dispersed and the external phase in each case as well as by the fine-particle character of the emulsion. Emulsifiers that can be used according to the invention are for example

addition products of 4 to 30 mol of ethylene oxide and/or 0 to 5 mol of propylene oxide with linear fatty alcohols having 8 to 22 C atoms, with fatty acids having 12 to 22 C atoms and with alkyl phenols having 8 to 15 C atoms in the alkyl group,

C₁₂-C₂₂ fatty acid monoesters and diesters of addition products of 1 to 30 mol of ethylene oxide with polyols having 3 to 6 carbon atoms, in particular with glycerol,

ethylene oxide and polyglycerol addition products with methyl glucoside fatty acid esters, fatty acid alkanol amides and fatty acid glucamides,

C₈-C₂₂ alkyl mono- and oligoglycosides and ethoxylated analogs thereof, wherein degrees of oligomerization of 1.1 to 5, in particular 1.2 to 2.0, and glucose as the sugar component are preferred,

mixtures of alkyl (oligo)glucosides and fatty alcohols, for example the commercially available product Montanov®68,

addition products of 5 to 60 mol of ethylene oxide with castor oil and hydrogenated castor oil,

partial esters of polyols having 3 to 6 carbon atoms with saturated fatty acids having 8 to 22 C atoms,

sterols. Sterols are understood to be a group of steroids which bear a hydroxyl group on C atom 3 of the steroid skeleton and are isolated from both animal tissue (zoosterols) and vegetable fats (phytosterols). Examples of zoosterols are cholesterol and lanosterol. Examples of suitable phytosterols are ergosterol, stigmasterol and sitosterol. Sterols known as mycosterols are also isolated from fungi and yeasts.

phospholipids. These are understood above all to be the glucose phospholipids which are obtained for example as lecithins or phosphatidyl cholines from for example egg yolk or plant seeds (e.g. soybeans),

fatty acid esters of sugars and sugar alcohols such as sorbitol,

polyglycerols and polyglycerol derivatives such as for example polyglycerol poly-12-hydroxystearate (commercial product Dehymuls® PGPH),

linear and branched fatty acids having 8 to 30 C atoms and Na, K, ammonium, Ca, Mg and Zn salts thereof.

The agents according to the invention contain the emulsifiers preferably in amounts from 0.1 to 25 wt. %, in particular 0.5 to 15 wt. %, relative to the total agent. The compositions according to the invention can preferably contain at least one non-ionogenic emulsifier having an HLB value of 8 to 18. Non-ionogenic emulsifiers having an HLB value of 10 to 15 can be particularly preferred according to the invention.

Depending on the nature of the agent according to the invention, it may be necessary for it also to contain at least one surfactant. This applies in particular in the case of skin cleaning agents and shampoos. However, other agents, such as for example hair rinses, hair masks and certain styling agents, in particular styling foams, can also contain surfactants.

Cationic surfactants can be used for example, as already described above as suitable care substances. With regard to the preferred cationic surfactants and the amounts used, the details given above apply correspondingly.

In addition to or in place of the cationic surfactants, the agents can contain further surfactants or emulsifiers, with both anionic and ampholytic and non-ionic surfactants and all types of known emulsifiers being suitable in principle. The group of ampholytic or amphoteric surfactants encompasses zwitterionic surfactants and ampholytes. The surfactants can already have an emulsifying action.

All anionic surface-active substances which are suitable for use on the human body are suitable in principle as anionic surfactants. These have the characterizing feature of a water-solubilizing anionic group such as for example a carboxylate, sulfate, sulfonate or phosphate group and a lipophilic alkyl group having approximately 8 to 30 C atoms. The molecule can additionally contain glycol or polyglycol ether groups, ester, ether and amide groups and hydroxyl groups. Examples of suitable anionic surfactants, each in the form of the sodium, potassium and ammonium salts as well as the mono-, di- and trialkanolammonium salts having 2 to 4 C atoms in the alkanol group, are

linear and branched fatty acids having 8 to 30 C atoms (soaps),

ether carboxylic acids of the formula R—O—(CH₂—CH₂O)ₓ—CH₂—COOH, in which R is a linear alkyl group having 8 to 30 C atoms and x=0 or 1 to 16,

acyl sarcosides having 8 to 24 C atoms in the acyl group,

acyl taurides having 8 to 24 C atoms in the acyl group,

acyl isethionates having 8 to 24 C atoms in the acyl group,

sulfosuccinic acid mono- and dialkyl esters having 8 to 24 C atoms in the alkyl group and sulfosuccinic acid monoalkyl polyoxyethyl esters having 8 to 24 C atoms in the alkyl group and 1 to 6 oxyethyl groups,

linear alkane sulfonates having 8 to 24 C atoms,

linear alpha-olefin sulfonates having 8 to 24 C atoms,

alpha-sulfo fatty acid methyl esters of fatty acids having 8 to 30 C atoms,

alkyl sulfates and alkyl polyglycol ether sulfates of the formula R—O(CH₂—CH₂O)ₓ—OSO₃H, in which R is a preferably linear alkyl group having 8 to 30 C atoms and x=0 or 1 to 12,

mixtures of surface-active hydroxyl sulfonates,

sulfated hydroxyalkyl polyethylene and/or hydroxyalkylene propylene glycol ethers,

sulfonates of unsaturated fatty acids having 8 to 24 C atoms and 1 to 6 double bonds,

esters of tartaric acid and citric acid with alcohols that are addition products of around 2 to 15 molecules of ethylene oxide and/or propylene oxide with fatty alcohols having 8 to 22 C atoms,

alkyl and/or alkenyl ether phosphates,

sulfated fatty acid alkylene glycol esters of formula (E1-II)

R⁷CO(AlkO)ₙSO₃M (E1-II)

in which R⁷CO denotes a linear or branched, aliphatic, saturated and/or unsaturated acyl residue having 6 to 22 C atoms, Alk denotes CH₂CH₂, CHCH₃CH₂ and/or CH₂CHCH₃, n denotes numbers from 0.5 to 5 and M denotes a cation such as are described in DE-OS 197 36 906,

amide ether carboxylic acids,

condensation products of C₈ to C₃₀ fatty alcohols with protein hydrolysates and/or amino acids and derivatives thereof, which are known to the person skilled in the art as protein fatty acid condensates, such as for example the Lamepon® types, Gluadin® types, Hostapon® KCG or Amisoft® types.

Preferred anionic surfactants are alkyl sulfates, alkyl polyglycol ether sulfates and ether carboxylic acids having 10 to 18 C atoms in the alkyl group and up to 12 glycol ether groups in the molecule, sulfosuccinic acid mono- and dialkyl esters having 8 to 18 C atoms in the alkyl group and sulfosuccinic acid monoalkyl polyoxyethyl esters having 8 to 18 C atoms in the alkyl group and 1 to 6 oxyethyl groups, monoglyceride sulfates, alkyl and alkenyl ether phosphates and protein fatty acid condensates.

Surface-active compounds classed as zwitterionic surfactants are those bearing at least one quaternary ammonium group and at least one —COO⁽⁻⁾ or —SO₃⁽⁻⁾ group in the molecule. Particularly suitable zwitterionic surfactants are the betaines such as N-alkyl-N,N-dimethylammonium glycinates, for example cocoalkyl dimethylammonium glycinate, N-acyl aminopropyl-N,N-dimethylammonium glycinates, for example cocoacylaminopropyl dimethylammonium glycinate, and 2-alkyl-3-carboxymethyl-3-hydroxyethyl imidazolines each having 8 to 18 C atoms in the alkyl or acyl group, and cocoacylaminoethyl hydroxyethyl carboxymethyl glycinate. A preferred zwitterionic surfactant is the fatty acid amide derivative known under the INCI name Cocamidopropyl Betaine.

Ampholytes are understood to be surface-active compounds which in addition to a C₈-C₂₄ alkyl or acyl group contain at least one free amino group and at least one —COOH or —SO₃H group in the molecule and are capable of forming internal salts. Examples of suitable ampholytes are N-alkyl glycines, N-alkyl propionic acids, N-alkyl aminobutyric acids, N-alkyl iminodipropionic acids, N-hydroxyethyl-N-alkyl amidopropyl glycines, N-alkyl taurines, N-alkyl sarcosines, 2-alkyl aminopropionic acids and alkyl aminoacetic acids, each having approximately 8 to 24 C atoms in the alkyl group. Particularly preferred ampholytes are N-cocoalkyl aminopropionate, cocoacylaminoethyl aminopropionate and C₁₂-C₁₈ acyl sarcosine.

Non-ionic surfactants contain as a hydrophilic group a polyol group, a polyalkylene glycol ether group or a combination of a polyol and polyglycol ether group, for example. Such compounds are for example

addition products of 2 to 50 mol of ethylene oxide and/or 1 to 5 mol of propylene oxide with linear and branched fatty alcohols having 8 to 30 C atoms, with fatty acids having 8 to 30 C atoms and with alkyl phenols having 8 to 15 C atoms in the alkyl group,

addition products of 2 to 50 mol of ethylene oxide and/or 1 to 5 mol of propylene oxide with linear and branched fatty alcohols having 8 to 30 C atoms, with fatty acids having 8 to 30 C atoms and with alkylphenols having 8 to 15 C atoms in the alkyl group, end-capped with a methyl or C₂ to C₆ alkyl residue, such as for example the types available under the commercial names Dehydol® LS, Dehydol® LT (Cognis),

C₁₂-C₃₀ fatty acid monoesters and diesters of addition products of 1 to 30 mol of ethylene oxide with glycerol,

addition products of 5 to 60 mol of ethylene oxide with castor oil and hydrogenated castor oil,

polyol fatty acid esters, such as for example the commercial product Hydagen® HSP (Cognis) or Sovermol types (Cognis),

alkoxylated triglycerides,

alkoxylated fatty acid alkyl esters of formula (E4-I)

R¹CO—(OCH₂CHR²)_{w}OR³ (E4-I)

in which R¹CO denotes a linear or branched, saturated and/or unsaturated acyl residue having 6 to 22 carbon atoms, R² denotes hydrogen or methyl, R³ denotes linear or branched alkyl residues having 1 to 4 carbon atoms and w denotes numbers from 1 to 20,

amine oxides,

sorbitan fatty acid esters and addition products of ethylene oxide with sorbitan fatty acid esters such as for example polysorbates,

sugar fatty acid esters and addition products of ethylene oxide with sugar fatty acid esters,

addition products of ethylene oxide with fatty acid alkanol amides and fatty amines,

sugar surfactants of the alkyl and alkenyl oligoglycoside type according to formula (E4-II),

R⁴O-[G]ₚ (E4-II)

in which R⁴ denotes an alkyl or alkenyl residue having 4 to 22 carbon atoms, G denotes a sugar residue having 5 or 6 carbon atoms and p denotes numbers from 1 to 10. They can be obtained by means of the relevant methods of preparative organic chemistry. The preferred alkyl and/or alkenyl oligoglycosides are thus alkyl and/or alkenyl oligoglucosides. The index value p in the general formula (E4-II) indicates the degree of oligomerization (DP), i.e. the distribution of mono- and oligoglycosides, and denotes a number between 1 and 10. The alkyl or alkenyl residue R⁴ can derive from primary alcohols having 4 to 11, preferably 8 to 10 carbon atoms. Alkyl oligoglucosides based on hydrogenated C_{12/14} coconut alcohol with a DP of 1 to 3 are preferred.

The alkylene oxide addition products with saturated linear fatty alcohols and fatty acids each containing 2 to 30 mol of ethylene oxide per mol of fatty alcohol or fatty acid have proved themselves as preferred non-ionic surfactants. Preparations having outstanding properties are likewise obtained if they contain fatty acid esters of ethoxylated glycerol as non-ionic surfactants. These compounds are characterized by the following parameters. The alkyl residue R contains 6 to 22 carbon atoms and can be both linear and branched. Primary linear aliphatic residues and aliphatic residues that are methyl-branched in the 2-position are preferred. Such alkyl residues are for example 1-octyl, 1-decyl, 1-lauryl, 1-myristyl, 1-cetyl and 1-stearyl. 1-Octyl, 1-decyl, 1-lauryl and 1-myristyl are particularly preferred. If oxo alcohols are used as starting materials, compounds having an odd number of carbon atoms in the alkyl chain dominate.

The compounds having alkyl groups which are used as the surfactant can each be uniform substances. It is generally preferable, however, to use native vegetable or animal raw materials as starting materials for these substances, such that mixtures of substances having differing alkyl chain lengths depending on the individual raw material are obtained.

The additional surfactants are generally used in amounts from 0.1 to 45 wt. %, preferably 0.5 to 30 wt. % and most particularly preferably from 0.5 to 25 wt. %, relative in each case to the total composition. The amount used depends substantially on the purpose of the agent according to the invention. If it is a shampoo or another cleaning agent, surfactant amounts above 45 wt. % are also usual.

The agents according to the invention can be formulated in all conventional forms for cosmetic agents, for example in the form of solutions, which can be applied to the hair as a pump or aerosol spray, in the form of creams, emulsions, waxes, gels or surfactant-containing foaming solutions or other preparations that are suitable for application on the hair.

Hair creams and hair gels generally contain structuring agents and/or thickening polymers, which serve to impart the desired consistency to the products. Structuring agents and/or thickening polymers are typically used in an amount from 0.1 to 10 wt. %, relative to the total product. Amounts from 0.5 to 5 wt. %, in particular 0.5 to 3 wt. %, are preferred. However, as the polymer combination used according to the invention has self-thickening properties, the addition of further structuring agents and/or thickening polymers is not absolutely necessary. The agents according to the invention preferably contain no further structuring agents and/or thickening polymers.

If the agent according to the invention is an aerosol product, it must contain a propellant. Propellants that are suitable according to the invention are for example N₂O, dimethyl ether, CO₂, air and alkanes having 3 to 5 carbon atoms, such as propane, n-butane, isobutane, n-pentane and isopentane, and mixtures thereof. Dimethyl ether, propane, n-butane, isobutane and mixtures thereof are preferred. The specified alkanes, mixtures of the specified alkanes or mixtures of the specified alkanes with dimethyl ether are preferably used as the sole propellant. However the invention expressly also encompasses the incorporation of propellants of the chlorofluorocarbon type, but in particular fluorocarbons. In the given spray device the sizes of the aerosol droplets or foam bubbles and the size distribution in each case can be adjusted by the ratio of propellant to the other constituents of the preparations.

The amount of propellant used varies according to the specific composition of the agent, the packaging used and the desired product type, for instance hair spray or hair foam. If conventional spray devices are used, aerosol foam products preferably contain the propellant in amounts from 1 to 35 wt. %, relative to the total product. Amounts from 2 to 30 wt. %, in particular from 3 to 15 wt. %, are particularly preferred. Aerosol sprays generally contain larger amounts of propellant. In this case the propellant is preferably used in an amount from 30 to 98 wt. %, relative to the total product. Amounts from 40 to 95 wt. %, in particular from 50 to 95 wt. %, are particularly preferred.

The aerosol products can be produced in the conventional manner. All constituents of the individual agent with the exception of the propellant are generally introduced into a suitable pressure-resistant container. This is then closed with a valve. Finally the desired amount of propellant is added using conventional techniques.

The invention therefore secondly provides the use of an agent according to the invention for temporarily shaping hair and/or for hair care.

The invention thirdly provides a method for treating keratin-containing fibers, in particular human hair, wherein an agent according to the invention is applied to the keratin-containing fibers.

All that has been stated in respect of the agents according to the invention applies with necessary alterations to the use according to the invention and to the method according to the invention.

### Examples

Unless otherwise specified, the quantities given below are percentages by weight.

The styling agents E1 to E4 according to the invention were produced in accordance with the table below:

TABLE 1

| Raw material (INCI name) | E1 | E2 | E3 | E4 |
|---|---|---|---|---|
| Aculyn ® 88 ¹ | 7.0 | 7.5 | 8.0 | 8.5 |
| Polymer (b) ² | 5.0 | 5.5 | 6.0 | 7.0 |
| Polyvinylpyrrolidone | 5.0 | 5.0 | 5.0 | 5.0 |
| 2-Amino-2-methyl-1-propanol | 1.0 | 1.25 | 1.5 | 1.75 |
| Glycerol | 1.0 | 1.0 | 1.0 | 1.0 |
| Propanediol-1,2 | 1.0 | 1.0 | 1.0 | 1.0 |
| Phenoxyethanol | 0.5 | 0.5 | 0.5 | 0.5 |
| Dye | 0.002 | 0.002 | 0.002 | 0.002 |
| Perfume | 0.10 | 0.10 | 0.10 | 0.20 |
| Ethanol 96%, denatured | — | — | — | 10.70 |
| Water, deionized | to 100 | to 100 | to 100 | to 100 |

¹ Copolymer of (meth)acrylic acid, (meth)acrylic acid ester and Steareth-20-methacrylic acid ester (28-30 wt. % solids in water; INCI name: Acrylates/Steareth-20 Methacrylate Crosspolymer) (Rohm and Haas),
² Linear tetrapolymer of methacrylic acid, hydroxyethyl methacrylate, methyl methacrylate and butyl acrylate

The agents were produced by conventional mixing of the raw materials listed in the table and used for hair styling by rubbing onto the surfaces of the hands and then applying to dry or wet hair.

The hair exhibited a soft shine, extreme and rapid hold and no flaking.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. An agent for treating keratin-containing fibers, in particular human hair, comprising in a cosmetically acceptable carrier—relative in each case to the weight of the agent—
(a) 1 to 20 wt. % of at least one crosslinked, amphiphilic, anionic polymer comprising at least one structural unit of formula (I) and at least one structural unit of formula (II), 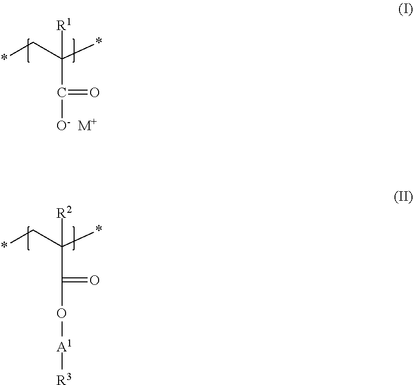
in which
R¹ and R² independently of one another denote a hydrogen atom or a methyl group,
R³ denotes a (C₈ to C₃₀) alkyl group,
M⁺ denotes a physiologically acceptable cation, and
A¹ denotes a *—(CH₂CH₂O)ₓ—* group in which x denotes an integer from 5 to 35, a *—(CH₂CHMeO)_{y}—* group in which y denotes an integer from 5 to 35 or a *—(CH₂CH₂O)ₓ—(CH₂CHMeO)_{y}—* group in which the sum of x+y
denotes an integer from 5 to 35 and x and y are greater than zero
and
(b) 1 to 20 wt. % of at least one uncrosslinked, anionic polymer comprising at least one structural unit of formula (III) and at least one structural unit of formula (IV), 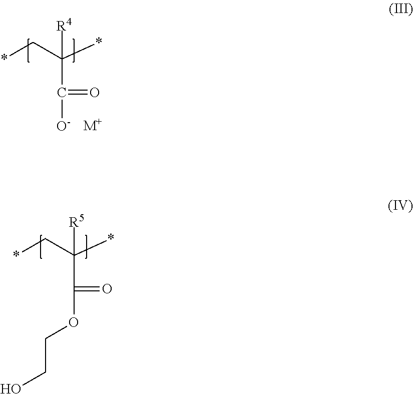
in which
R⁴ and R⁵ independently of one another denote a hydrogen atom or a methyl group, and
M⁺ denotes a physiologically acceptable cation, and
(c) 1 to 40 wt. % of at least one fixing non-ionic polymer from the group consisting of
(c1) non-ionic polymers having at least one structural element of formula (N5) 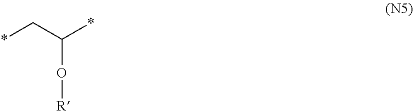
in which R′ denotes a hydrogen atom, an alkyl group or an alkanoyl group,
(c2) homopolymers and non-ionic copolymers of N-vinylpyrrolidone, and
(c3) non-ionic copolymers of isobutene.

2. The agent according to claim 1, wherein according to formula (II) A¹ denotes a *—(CH₂CH₂O)ₓ—* group in which x denotes an integer from 5 to 35.

3. The agent according claim 1, wherein according to formula (II) R² denotes a methyl group.

4. The agent according to claim 1, wherein the uncrosslinked, anionic polymer (b) additionally includes at least one structural unit of formula (V), 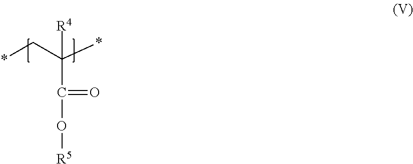
in which
R⁴ denotes a hydrogen atom or a methyl group, and
R⁵ denotes a (C₂ to C₄) alkyl group.

5. The agent according to claim 1, wherein the uncrosslinked, anionic polymer (b) is a linear tetrapolymer consisting of methacrylic acid, hydroxyethyl methacrylate, methyl methacrylate and butyl acrylate.

6. The agent according to claim 1, wherein the fixing non-ionic polymer (c) comprises at least one polymer selected from the group consisting of
polyvinylpyrrolidone,
copolymers of N-vinylpyrrolidone and vinyl esters of carboxylic acids having 2 to 18 carbon atoms, in particular of N-vinylpyrrolidone and vinyl acetate,
copolymers of N-vinylpyrrolidone and N-vinylimidazole and methacrylamide,
copolymers of N-vinylpyrrolidone and N-vinylimidazole and acrylamide,
copolymers of N-vinylpyrrolidone with N,N-di(C₁ to C₄) alkylamino-(C₂ to C₄)-alkylacrylamide, and
copolymers of N-vinylpyrrolidone with N,N-di(C₁ to C₄) alkylamino-(C₂ to C₄)-alkylacrylamide.

7. The agent according to claim 1, wherein the agent comprises—relative to its weight—
the crosslinked, amphiphilic, anionic polymers (a) in an amount from 1.5 to 17.5 wt. %,
the uncrosslinked, anionic polymers (b) in an amount from 1.5 to 17.5 wt. %, and
the fixing non-ionic polymers (c) in an amount from 1.5 to 35 wt.

8. The agent according to claim 1, wherein the agent comprises the uncrosslinked, amphiphilic, anionic polymers (b) in an amount from 0.1 wt. % to 5.0 wt. %, relative in each case to the weight of the agent.

9. The agent according to claim 1, wherein the crosslinked, amphiphilic, anionic polymers (a) and the uncrosslinked, anionic polymers (b) are used in a weight ratio from 1 to 5 to 5 to 1.

10. A method for treating keratin-containing fibers, in particular human hair, comprising:
applying the agent according to claim 1 to the keratin-containing fibers.
